(19) United States
(12) Patent Application Publication
(10) Pub. No.: US 2022/0330889 A1
(43) Pub. Date: Oct. 20, 2022

(54) ARTIFICIAL INTELLIGENCE INFRARED THERMAL IMAGING BRA SYSTEM WITH BREAST DISEASE DETECTING FUNCTION

(71) Applicant: Peking Union Medical College Hospital, Chinese Academy of Medical Sciences, Beijing (CN)

(72) Inventors: Xuefei WANG, Beijing (CN); Guanyu ZHOU, Beijing (CN); Qiang SUN, Beijing (CN); Jidong ZHAI, Beijing (CN); Likun HUNAG, Taiyuan (CN); Yidong ZHOU, Beijing (CN); Guochao ZHANG, Beijing (CN); Feng MAO, Beijing (CN); Yan LIN, Beijing (CN); Songjie SHEN, Beijing (CN); Xiaohui ZHANG, Beijing (CN); Xuejing WANG, Beijing (CN)

(73) Assignee: Peking Union Medical College Hospital, Chinese Academy of Medical Sciences, Beijing (CN)

(21) Appl. No.: 17/858,205

(22) Filed: Jul. 6, 2022

Related U.S. Application Data
(63) Continuation of application No. PCT/CN2021/130646, filed on Nov. 15, 2021.

(30) Foreign Application Priority Data
Dec. 21, 2020 (CN) 202011518638.6

(51) Int. Cl. A61B 5/00; A41C 3/00; A61B 5/01

(52) U.S. Cl. CPC A61B 5/6804; A41C 3/0064; A41C 3/0028; A61B 5/015; A61B 5/0091

(57) ABSTRACT

Disclosed is an artificial intelligence infrared thermal imaging bra system with breast disease detecting function. The system includes a side support, two chest support layers and two shoulder straps; the two shoulder straps are fixed at both ends of the side support, and the two chest support layers are symmetrically fixed between the two shoulder straps at the top of the side support; the two chest support layers are internally provided with sliding mechanisms that are adjustable according to different chests of users; a rotating mechanism for driving the sliding mechanisms to move is arranged between the two chest support layers at the top of the side support, and the rotating mechanism and the sliding mechanisms are in transmission connection; one end of the side support is fixed with a sub-buckle, and the other end of the side support is provided with a stretching mechanism.

### CROSS-REFERENCE TO RELATED APPLICATIONS

The present application is a continuation of International Application No. PCT/CN2021/130646, filed on Nov. 15, 2021, which claims priority to Chinese Patent Application No. 202011518638.6, filed on Dec. 21, 2020, the contents of which are hereby incorporated by reference.

### TECHNICAL FIELD

The application relates to the technical field of medical instruments, and particularly to an artificial intelligence infrared thermal imaging bra system with a breast disease detecting function.

### BACKGROUND

Breast cancer is classified as the most commonly reported cancer in the world. Both men and women have been reported suffering this kind of cancer. Cancer cells are often screened out in ducts and lobules. There are many causes for the breast cancer, some of which are still unclear. However, one of the confirmed causes for the breast cancer is abnormal cell development. Some genes are responsible for cell division and proliferation. However, for some reasons, abnormal growth occurs, and leads to the rapid growth and diffusion of cancer cells. Such growth depends on the supply of a large amount of blood. Cancer cells may spread to other parts. Breast cancer cells are transferred through lymph or blood. In the latter case, breast cancer is considered to be in an advanced stage, and the prognosis of an advanced breast cancer is dissatisfactory.

Therefore, early diagnosis and treatment of breast cancer is particularly important. Thermal imaging detection is one of the existing detection methods of breast diseases. However, all kinds of detection methods of breast diseases, including thermal imaging detection, require examiners to go to the hospital, which takes a long time to queue up and wait for results. Therefore, an artificial intelligence infrared thermal imaging bra system with the breast disease detecting function is proposed.

### SUMMARY

The objective of the application is to provide a convenient artificial intelligence infrared thermal imaging bra system with the breast disease detecting function, which can detect breast diseases at home, so as to solve the problems raised in the above background.

In order to achieve the above objective, the application provides the following technical scheme: an artificial intelligence infrared thermal imaging bra system with the breast disease detecting function includes a side support, two chest support layers and two shoulder straps; the two shoulder straps are fixed at both ends of the side support, and the two chest support layers are symmetrically fixed between the two shoulder straps at the top of the side support; the two chest support layers are internally provided with sliding mechanisms which can be adjusted according to different chests of users; a rotating mechanism for driving the sliding mechanisms to move is arranged between the two chest support layers at the top of the side support, and the rotating mechanism and the sliding mechanisms are in transmission connection; one end of the side support is fixed with a sub-buckle, and the other end of the side support is provided with a stretching mechanism which could make adjustment for users with different busts in conjunction with the sub-buckle.

In an embodiment, the rotating mechanism includes two connecting plates, a turntable, gears and racks; the two connecting plates are fixed at ends of the two chest support layers away from the two shoulder straps, the turntable is rotatably connected between the two connecting plates, the gears are fixed on both sides of the turntable through a drive shaft passing through the two connecting plates, and the racks are meshed and connected with bottoms of the gears.

In an embodiment, the sliding mechanisms include supporting plates, chutes, sliding sleeves and fiber columns; the supporting plates are slidably connected inside the two chest support layers, and sides of the supporting plates close to the gears are fixedly connected with the racks; the surfaces of the two chest support layers are symmetrically provided with the chutes, and the fiber columns are fixed inside the chutes; the sliding sleeves are fixed at the bottoms of the supporting plates corresponding to the fiber columns, the sliding sleeves are slidably sleeved on the surfaces of the fiber columns, and the sliding sleeves are in sliding connection with the chutes.

In an embodiment, protective pads are fixed on sides of the supporting plates away from the two chest support layers.

In an embodiment, the stretching mechanism includes a mother buckle, a rotating roller and a telescopic belt; the rotating roller is rotatably connected to one end of the side support; one end of the telescopic belt is fixed on the rotating roller, the other end of the telescopic belt is fixed with the mother buckle, and the sub-buckle is bonded with the mother buckle.

Compared with the prior art, the application has the following beneficial effects.

In the application, under the cooperation of the rotating mechanism and the adjusting mechanism, the turntable drives the gears on both sides to rotate and mesh with the racks. With the meshing transmission between the gears and the racks, the supporting plates slide along the two chest support layers, while the sliding sleeves slide along the direction of the fiber columns and the chutes in an auxiliary way and play a guiding role, so as to make the supporting plates hold up the chest of a tester. Therefore, adaptive adjustment for different chest sizes of different wearers is realized, which facilitates the holding of the chest and taking pictures.

Under the action of the stretching mechanism, covering the side support under the skin of the user's chest, pulling the mother buckle to stretch the telescopic belt under the winding action of the rotating roller, and adjusting adaptively according to different busts, so as to make the bra more comfortable to wear. Therefore, self-adaptive adjustment function of users with different bust sizes is realized, and the bra is more comfortable and convenient to wear.

After wearing the bra of the system, using the infrared thermal imaging camera of the system to connect with a mobile phone and take pictures of the breasts in the required environment; interpreting the infrared photos by an artificial intelligence image reading system included in the system, and then giving screening results. The application realizes e convenient breast disease detecting at home and provides the greatest possibility for the early detection of breast diseases.

### BRIEF DESCRIPTION OF THE FIGURES

FIG. 1 is a schematic diagram of the overall structure according to the application.

FIG. 2 is one of the schematic diagrams of the sliding mechanism according to the application.

FIG. 3 is a structural schematic diagram of the rotating mechanism according to the application.

FIG. 4 is a schematic diagram of the connection between the rotating mechanism and the sliding mechanism according to the application.

FIG. 5 is a schematic structural diagram of the stretching mechanism according to the application.

FIG. 6 is a schematic diagram of a thermal imaging process according to the application.

### DETAILED DESCRIPTION OF THE EMBODIMENTS

The technical schemes in the embodiments of the application will be clearly and completely described with reference to the drawings in the embodiments of the application. Obviously, the described embodiments are only part of the embodiments of the application, not all of them. Based on the embodiments in the application, all other embodiments obtained by ordinary technicians in the field without creative work are within the protection scope of the application.

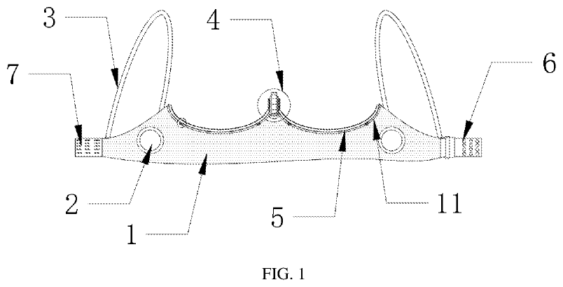

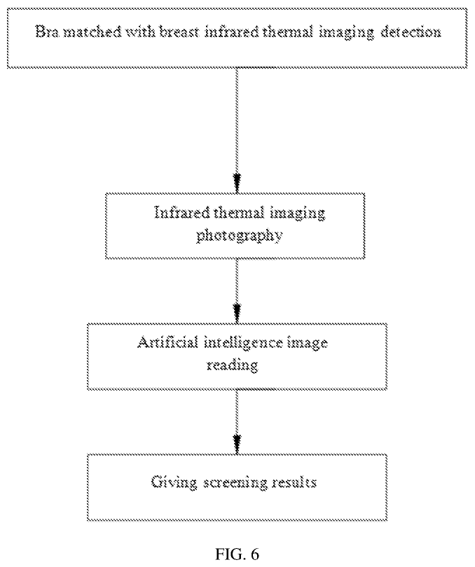

With reference to FIG. 1 and FIG. 6, the illustrated artificial intelligence infrared thermal imaging bra system with the breast disease detecting function includes a side support 1, two chest support layers 11 and two shoulder straps 3; the two shoulder straps 3 are fixed at both ends of the side support 1, the two chest support layers 11 are symmetrically fixed between the two shoulder straps 3 at the top of the side support 1, and the side support 1 and the two shoulder straps 3 are mainly made of 88% mulberry silk. When the device is used at home to detect breast diseases, a tester wears the bra on the body, installs the infrared thermal imager on a camera of a mobile phone to take pictures, the infrared photos is interpreted by an artificial intelligence image reading system contained in the system, and then the screening results. The artificial intelligence image reading system has the same working principle as the existing database when searching matching results.

With reference to FIG. 1, the two chest support layers 11 are internally provided with sliding mechanisms 5 which can be adjusted according to different chests of users; a rotating mechanism 4 for driving the sliding mechanisms 5 to move is arranged between the two chest support layers 11 at the top of the side support 1, and the rotating mechanism 4 and the sliding mechanisms 5 are in transmission connection; one end of the side support 1 is fixed with a sub-buckle 7, and the other end of the side support 1 is provided with a stretching mechanism 6 which can cooperate with the sub-buckle 7 to adjust users with different busts. With the cooperation of the rotating mechanism 4 and an adjusting mechanism, the adaptive adjustment for different wearers' chest sizes is realized, and it is convenient to hold up the chest and take pictures. With the stretching mechanism 6, users may make self-adaptive adjustment with different bust sizes, making the bra more comfortable and convenient to wear.

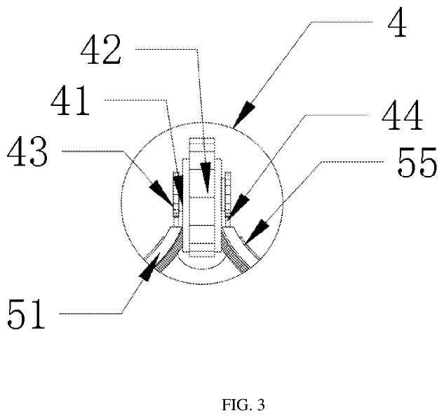

With reference to FIG. 3, the rotating mechanism 4 includes two connecting plates 41, a turntable 42, gears 43 and racks 44; the two connecting plates 41 are fixed at ends of the two chest support layers 11 away from the two shoulder straps 3, the turntable 42 is rotatably connected between the two connecting plates 41, the gears 43 are fixed on both sides of the turntable 42 through a drive shaft passing through the two connecting plates 41, and the racks 44 are meshed and connected with bottoms of the gears 43. Rotate the turntable 42, the turntable 42 drives the gears 43 on both sides to rotate and mesh with the racks 44.

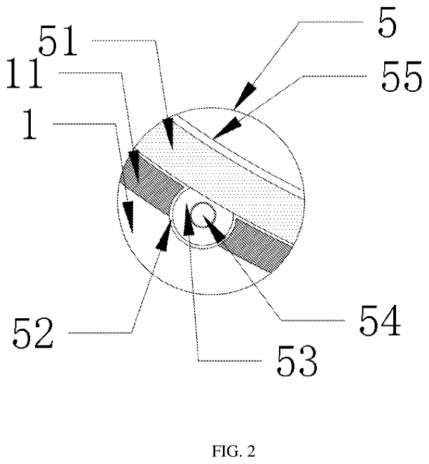

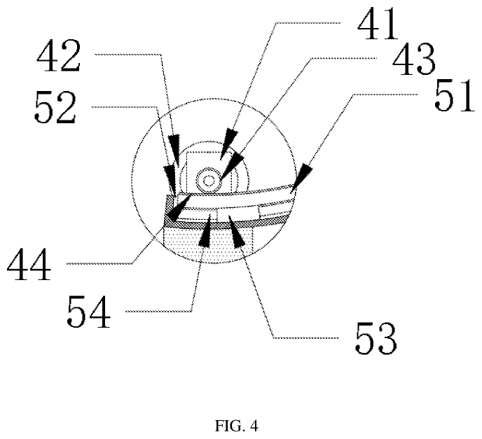

With reference to FIG. 2 and FIG. 4, the sliding mechanisms 5 include supporting plates 51, chutes 52, sliding sleeves 53 and fiber columns 54; the supporting plates 51 are slidably connected inside the two chest support layers 11, and sides of the supporting plates 51 close to the gears 43 are fixedly connected with the racks 44; the surfaces of the two chest support layers 11 are symmetrically provided with the chutes 52, and the fiber columns 54 are fixed inside the chutes 52; the sliding sleeves 53 are fixed at the bottoms of the supporting plates 51 corresponding to the fiber columns 54; the sliding sleeves 53 are slidably sleeved on the surfaces of the fiber columns 54, and the sliding sleeves 53 are in sliding connection with the chutes 52. Under the action of meshing transmission between the gears 43 and the racks 44, the supporting plates 51 slide along the two chest support layers 11, while the sliding sleeves 53 slide along the direction of the fiber columns 54 and the chutes 52 in an auxiliary way and play a guiding role.

With reference to FIG. 2 and FIG. 3, protective pads 55 are fixed on sides of the supporting plates 51 away from the two chest support layers 11, so as to reduce the discomfort of the contact between the supporting plates 51 and the skin of the user's chest.

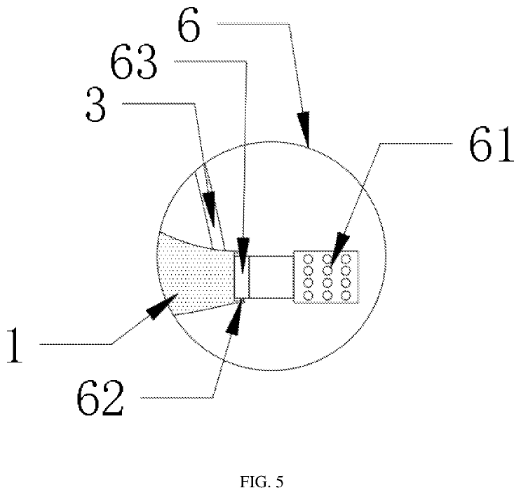

With reference to FIG. 1 and FIG. 5, the stretching mechanism 6 includes a mother buckle 61, a rotating roller 62 and a telescopic belt 63; the rotating roller 62 is rotatably connected to one end of the side support 1; one end of the telescopic belt 63 is fixed on the rotating roller 62, the other end of the telescopic belt 63 is fixed with the mother buckle 61, and the sub-buckle 7 is bonded with the mother buckle 61. When wearing the bra, the side support 1 is covered under the skin of the user's chest, the mother buckle 61 is pulled to stretch the telescopic belt 63 under the winding action of the rotating roller 62, and adjustment is made adaptively according to different bust sizes, so as to make the bra more comfortable to wear. Torsion springs are installed at both ends of the rotating roller 62 to assist in retracting the telescopic belt 63.

In this scheme, when wearing the bra, the side support 1 is covered under the skin of the user's chest, the mother buckle 61 is pulled to stretch the telescopic belt 63 under the winding action of the rotating roller 62, and adjustment is made adaptively according to different bust sizes, so as to make the bra more comfortable to wear. The turntable 42 drives the gears 43 on both sides to rotate and mesh with the racks 44. Under the action of meshing transmission between the gears 43 and the racks 44, the supporting plates 51 slide along the two chest support layers 11, while the sliding sleeves 53 slide along the direction of the fiber columns 54 and the chutes 52 in an auxiliary way and play a guiding role. After wearing, the infrared thermal imager is installed on a camera of a mobile phone to take pictures of a tester, interpret the infrared photos by an artificial intelligence image reading system contained in the system, and give the screening results. The breast disease detection is connected with the bra, and with the assistance of artificial intelligence image reading, the breast diseases, like blood pressure measurement with a sphygmomanometer, can be detected by everyone at home every day.

It should be noted that in this paper, relational terms such as first and second are only used to distinguish one entity or operation from another entity or operation, and do not necessarily require or imply any such actual relationship or order between these entities or operations. Furthermore, the terms “including”, “comprising” or any other variation thereof are intended to cover non-exclusive inclusion, so that a process, method, article or equipment including a series of elements includes not only those elements, but also other elements not explicitly listed, or elements inherent to such process, method, article or equipment.

Although the embodiments of the application have been shown and described, it will be understood by those of ordinary skill in the art that various changes, modifications, substitutions and modifications could be made to these embodiments without departing from the principles and spirit of the application, and the scope of the application is defined by the claims and their equivalents.

What is claimed is:

1. An artificial intelligence infrared thermal imaging bra system with a breast disease detecting function, comprising a side support, two chest support layers and two shoulder straps;
wherein the two shoulder straps are fixed at both ends of the side support, and the two chest support layers are symmetrically fixed between the two shoulder straps at a top of the side support; the two chest support layers are internally provided with sliding mechanisms that are adjustable according to different chests of users; a rotating mechanism for driving the sliding mechanisms to move is arranged between the two chest support layers at the top of the side support, and the rotating mechanism and the sliding mechanisms are in transmission connection; one end of the side support is fixed with a sub-buckle, and the other end of the side support is provided with a stretching mechanism that cooperates with the sub-buckle to adjust users with different busts.

2. The artificial intelligence infrared thermal imaging bra system according to claim 1, wherein the rotating mechanism comprises two connecting plates, a turntable, gears and racks; the two connecting plates are fixed at ends of the two chest support layers away from the two shoulder straps, the turntable is rotatably connected between the two connecting plates, the gears are fixed on both sides of the turntable through a drive shaft passing through the two connecting plates, and the racks are meshed and connected with bottoms of the gears.

3. The artificial intelligence infrared thermal imaging bra system according to claim 2, wherein the sliding mechanisms comprise supporting plates, chutes, sliding sleeves and fiber columns; the supporting plates are slidably connected inside the two chest support layers, and sides of the supporting plates close to the gears are fixedly connected with the racks; surfaces of the two chest support layers are symmetrically provided with the chutes, and the fiber columns are fixed inside the chutes; the sliding sleeves are fixed at the bottoms of the supporting plates corresponding to the fiber columns, the sliding sleeves are slidably sleeved on the surfaces of the fiber columns, and the sliding sleeves are in sliding connection with the chutes.

4. The artificial intelligence infrared thermal imaging bra system according to claim 3, wherein protective pads are fixed on sides of the supporting plates away from the two chest support layers.

5. The artificial intelligence infrared thermal imaging bra system according to claim 1, wherein the stretching mechanism comprises a mother buckle, a rotating roller and a telescopic belt; the rotating roller is rotatably connected to one end of the side support; one end of the telescopic belt is fixed on the rotating roller, the other end of the telescopic belt is fixed with the mother buckle, and the sub-buckle is bonded with the mother buckle.
